SEQUENCE LISTING (5 sequences)

SEQ ID NO: 1 - protein, 217 aa, Halothermothrix orenii
MIEAVIFDMDGVIINSEPIHYKVNQIIYEKLGIKVPRSEYNTFIGKSNTDIWSFLKRKYNLKESVSSLIEKQISGNIKYLKSHEVNPIPGVKPLLDELSEKQITTGLASSSPEIYIETVLEELGLKSYFKVTVSGETVARGKPEPDIFEKAARILGVEPPHCVVIEDSKNGVNAAKAAGMICIGYRNEESGDQDLSAADVVVDSLEKVNYQFIKDLI

SEQ ID NO: 2 - protein, 809 aa, Thermus
MPLLPEPLSGLKELAYNLWWSWNPEAAELFQEIDPSLWKRFRGNPVKLLLEADPGRLEGLAATSYPARVGAVVEALRAYLREREEKQGPLVAYFSAEYGFHSSLPIYSGGLGVLAGDHVKAASDLGLNLVGVGIFYHEGYFHQRLSPEGVQVEVYETLHPEELPLYPVQDREGRPLRVGVEFPGRTLWLSAYRVQVGAVPVYLLTANLPENTPEDRAITARLYAPGLEMRIQQELVLGLGGVRLLRALGLAPEVFHMNEGHSAFLGLERVRELVAEGHPFPVALELARAGALFTTHTPVPAGHDAFPLELVERYLGGFWERMGTDRETFLSLGLEEKPWGKVFSMSNLALRTSAQANGVSRLHGEVSREMFHHLWPGFLREEVPIGHVTNGVHTWTFLHPRLRRHYAEVFGPEWRKRPEDPETWKVEALGEEFWQIHKDLRAELVREVRTRLYEQRRRNGESPSRLREAEKVLDPEALTIGFARRFATYKRAVLLFKDPERLRRLLHGHYPIQFVFAGKAHPKDEPGKAYLQELFAKIREYGLEDRMVVLEDYDMYLARVLVHGSDVWLNTPRRPMEASGTSGMKAALNGALNLSVLDGWWAEAYNGKNGFAIGDERVYESEEAQDMADAQALYDVLEFEVLPLFYAKGPEGYSSGWLSMVHESLRTVGPRYSAARMVGDYLEIYRRGGAWAEAARAGQEALAAFHQALPALQGVTLRAQVPGDLTLNGVPMRVRAFLEGEVPEALRPFLEVQLVVRRSSGHLEVVPMRPGPDGYEVAYRPSRPGSYAYGVRLALRHPITGHVAWVRWA

SEQ ID NO: 3 - protein, 581 aa, Caldibacillus debilis
MEWKQRAERWLRFENLDPELKKQLEEMAKDEKKLEDLFYKYLEFGTGGMRGEIGPGTNRINIYTVRKASEGLARFLLASGGEEKAKQGVVIAYDSRRKSREFALETAKTVGKHGIKAYVFESLRPTPELSFAVRYLHAAAGVVITASHNPPEYNGYKVYGEDGGQLTPKAADELIRYVYEVEDELSLTVPGEQELIDRGLLQYIGENIDLAYIEKLKTIQLNRDVILNGGKDLKIVFTPLHGTAGQLVQTGLREFGFQNVYVVKEQEQPDPDFSTVKSPNPEEHEAFEIAIRYGKKYDADLIMGTDPDSDRLGIVVKNGQGDYVVLTGNQTGAILLYYLLSQKKEKGMLVRNSAVLKTIVTSELGRAIASDFGVETIDTLTGFKFIGEKIKEFKETGSHVFQFGYEESYGYLIGDFVRDKDAIQAALFAAEAAAYYKAQGKSLYDVLMEIYKKYGFYKESLRSITLKGKDGAEKIRAIMDAFRQNPPEEVSGIPVAITEDYLTQKRVDKAAGQTTPIHLPKSNVLKYYLADESWFCIRPSGTEPKCKFYFAVRGDSEAQSEARLRQLETNVMAMVEKILQK

SEQ ID NO: 4 - protein, 415 aa, Thermus thermophilus
MLRLDTRFLPGFPEALSRHGPLLEEARRRLLAKRGEPGSMLGWMDLPEDTETLREVRRYREANPWVEDFVLIGIGGSALGPKALEAAFNESGVRFHYLDHVEPEPILRLLRTLDPRKTLVNAVSKSGSTAETLAGLAVFLKWLKAHLGEDWRRHLVVTTDPKEGPLRAFAEREGLKAFAIPKEVGGRFSALSPVGLLPLAFAGADLDALLMGARKANETALAPLEESLPLKTALLLHLHRHLPVHVFMVYSERLSHLPSWFVQLHDESLGKVDRQGQRVGTTAVPALGPKDQHAQVQLFREGPLDKLLALVIPEAPLEDVEIPEVEGLEAASYLFGKTLFQLLKAEAEATYEALAEAGQRVYALFLPEVSPYAVGWLMQHLMWQTAFLGELWEVNAFDQPGVELGKVLTRKRLAG

SEQ ID NO: 5 - protein, 511 aa, Anaerolinea thermophila
MSLFKRASGILLHPTSLPGPDGIGDLGPEAYRWVNFLAESGCSLWQILPLGPTGFGDSPYQCFSAFAGNPYLVSPALLLDEGLLTSEDLADRPEFPASRVDYGPVIQWKLTLLDRAYVRFKRSTSQKRKAAFEAFKEEQRAWLLDFSLFMAIKEAHGGASWDYWPEPLRKRDPEALNAFHRAHEVDVERHSFRQFLFFRQWQALRQYAHEKGVQIIGDVPIFVAYDSADVWSHPDLFYLDETGKPTVVAGVPPDYFSATGQLWGNPLYRWDYHRETGFAWWLERLKATFAMVDIVRLDHFRGFAGYWEVPYGMPTAEKGRWVPGPGIALFEAIRNALGGLPIIAEDLGEITPDVIELREQLGLPGMKIFQFAFASDADDPFLPHNYVQNCVAYTGTHDNDTAIGWYNSAPEKERDFVRRYLARSGEDIAWDMIRAVWSSVAMFAIAPLQDFLKLGPEARMNYPGRPAGNWGWRYEAFMLDDGLKNRIKEINYLYGRLPEHMKPPKVVKKWT

(19) United States
(12) Patent Application Publication
(10) Pub. No.: US 2022/0235386 A1
(43) Pub. Date: Jul. 28, 2022

(54) ENZYMATIC PRODUCTION OF FRUCTOSE

(71) Applicant: BONUMOSE, INC, Charlottesville, VA (US)

(72) Inventor: Daniel Joseph WICHELECKI, Charlottesville, VA (US)

(21) Appl. No.: 17/613,315

(22) PCT Filed: Jun. 1, 2020
(86) PCT No.: PCT/US2020/000022

§ 371 Date: Nov. 22, 2021

Related U.S. Application Data
(60) Provisional application No. 62/855,397, filed on May 31, 2019.

(51) Int. Cl. C12P 19/02

(52) U.S. Cl. CPC C12P 19/02

(57) ABSTRACT

Production of fructose from saccliarides by processes, in which fructose 6-phosphate phosphatase (F6PP) catalyzes the conversion of fructose 6-phosphate (F6P) to a fructose, in the presence of one or more divalent cations, Mg²⁺, Zn²⁺, Ca²⁺, Co²⁺, and Mn²⁺, are disclosed herein.

### FIELD OF THE INVENTION

The invention relates to processes for the enzymatic production of fructose.

### BACKGROUND

Fructose is a simple ketonic monosaccharide found in many plants, where it is often bonded to glucose to form the disaccharide, sucrose. Commercially, fructose is derived from sugar cane, sugar beets, and maize. The primary reason that fructose is used commercially in foods and beverages, besides its low cost, is its high relative sweetness. It is the sweetest of all naturally occurring carbohydrates. Fructose is also found in the manufactured sweetener, high-fructose corn syrup (HFCS), which is produced by treating corn syrup with enzymes, converting glucose into fructose with yields limited by equilibrium. (en.wikipedia.org/wiki/Fructose#Physical_and_functional_properties--accessed 3/7/18). Alternatively, fructose can be used as a precursor for the production of the alternative sweetener allulose (see, for example, WO2016160573A1, WO2015032761A1, WO2014049373A1) or as a precursor to hydroxymethylfurfural which can then be turned into various useful chemicals such as 2,5-furandicarboxylic acid or 2,5-dimethylfuran (en.wikipedia.org/wiki/Hydroxymethylfurfural). These applications demand high purity fructose, whereas the sweetener application can be utilized as either high purity or low purity fructose.

There is a need to develop cost-effective synthetic pathways for high-yield, high purity production of the fructose where at least one step of the processes involves an energetically favorable chemical reaction. Furthermore, there is a need for production processes where the process steps can be conducted in one tank or bioreactor and/or where costly separation steps are avoided or eliminated. There is also a need for high-yield, high purity processes of fructose production that can be conducted at a relatively low concentration of phosphate, where phosphate can be recycled and/or the process does not require using adenosine triphosphate (ATP) as an added source of phosphate.

Enzymatic processes for preparing fructose are provided in PCT Pat. Appl. No. WO 2018/169957, which is incorporated by reference in its entirety. It is desirable to improve fructose yields and enzyme activity to reduce the cost of the process. In that regard, as described below, the use of certain divalent cations in processes for producing fructose result in significant improvements fructose 6-phosphate phosphatase activity and fructose yield.

### SUMMARY OF THE INVENTION

The inventions described herein generally relate to improved processes for the enzymatic production of fructose. More particularly, a process of the invention includes a step of converting fructose 6-phosphate to fructose by a reaction catalyzed by fructose 6-phosphate phosphatase (F6PP) in the presence of one or more divalent cations, including, for example, Mg²⁺, Zn²⁺, Ca²⁺, Co²⁺, Mn²⁺, and combinations thereof. Accordingly, a process according to of the invention can include a step of converting fructose 6-phosphate to fructose by a reaction catalyzed by F6PP in the presence of Mg' and a divalent cation selected from the group consisting of Zn²⁺, Ca²⁺, Co²⁺, Mn²⁺, and combinations thereof.

### BRIEF DESCRIPTION OF THE FIGURES

FIG. 1 is a schematic diagram showing an enzymatic pathway converting starch or its derived products to fructose. The following abbreviations are used: IA, isoamylase; PA, pullulanase; aGP, alphaglucan phosphorylase or starch phosphorylase; MP, maltose phosphorylase; PGM, phosphoglucomutase; PPGK, polyphosphate glucokinase; PGI, phosphoglucoisomerase; F6PP, fructose 6-phosphate phosphatase.

FIG. 2 is a schematic diagram showing an enzymatic pathway converting sucrose to fructose. The following abbreviations are used: SP, sucrose phosphorylase; PGM, phosphoglucomutase; PGI, phosphoglucoisomerase; F6PP, fructose 6-phosphate phosphatase.

FIG. 3 shows a chromatogram of a study of the effect of various divalent cations, with magnesium, on the yield of fructose from maltodextrin. Fructose is seen at a retention time of ˜19 minutes, starting material between 8 and 11.5 minutes, maltotriose at 11.75 min, maltose at 12.75 minutes, and glucose at 14 minutes. No additional divalent cation=blue; calcium=red; cobalt=green; manganese=pink; copper=gold; and zinc=purple.

FIG. 4 shows a chromatogram of a study of different concentrations of Co2+and Mn2+, with magnesium, in the reaction mixture. 1 mM cobalt=blue; 0.5 mM cobalt=red; 0.1 mM cobalt=green; 1 mM manganese=pink; 0.5 mM manganese=gold; and 0.1 mM manganese=purple.

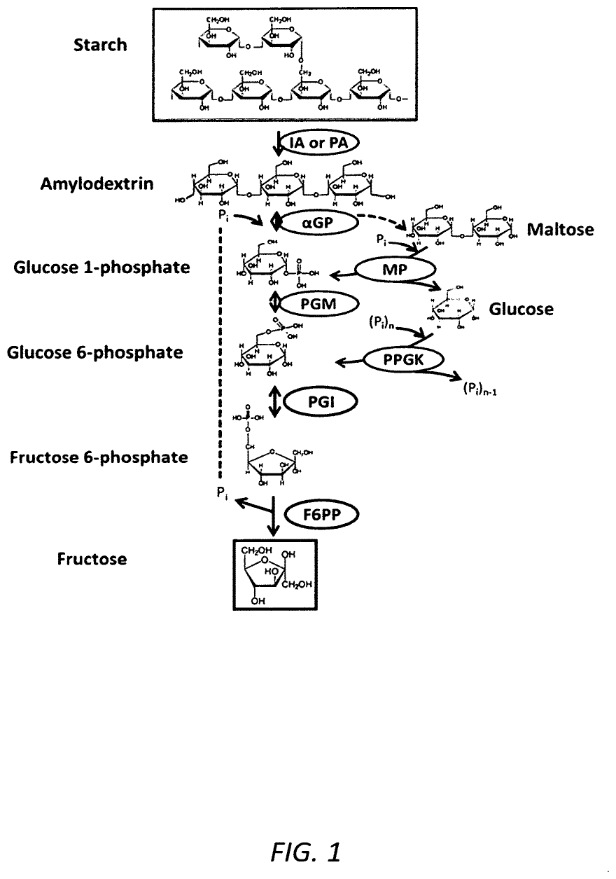

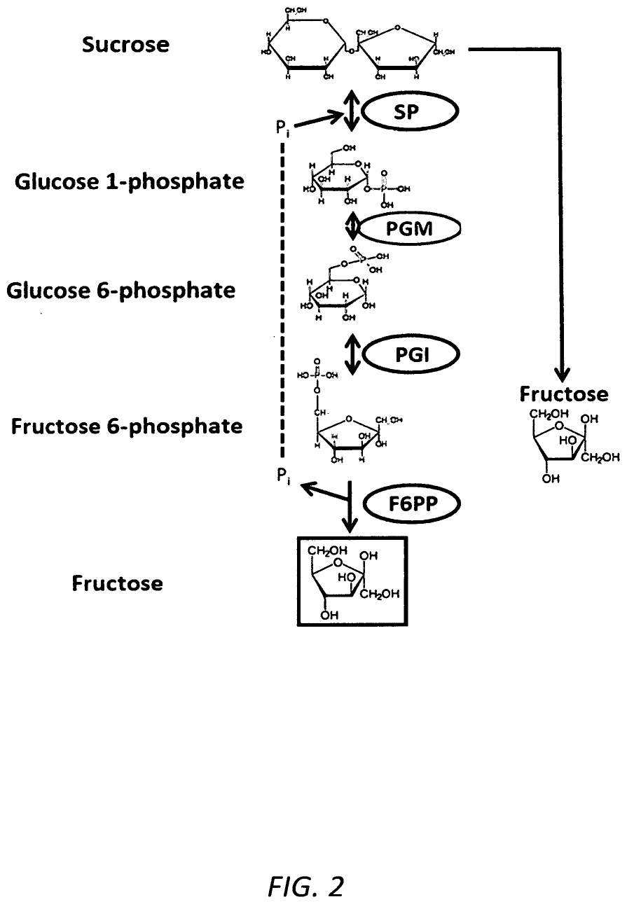

### DETAILED DESCRIPTION

The inventions described herein provide improved enzymatic pathways, or processes, for producing fructose with a high product yield, while also decreasing fructose production costs. Also described herein is fructose produced by these process. Improved processes of the invention for the enzymatic production of fructose include a step of converting fructose 6-phosphate to fructose catalyzed by fructose 6-phosphate phosphatase (F6PP), in the presence of a divalent cation. For example, a process of the invention for converting F6P to fructose can be performed in the presence of one or more divalent cations selected from the group consisting of Mg²⁺, Zn²⁺, Ca²⁺, Co', Mn²⁺, and combinations thereof. Some embodiments include a step of converting fructose 6-phosphate to fructose catalyzed by F6PP in the presence of Mg²⁺ and a divalent cation selected from the group consisting of Zn²⁺, Ca²⁺, Co²⁺, Mn²⁺, and combinations thereof. Without wishing to be bound by a particular theory, it is believed that the divalent cation stabilizes the F6PP, resulting in higher activity and greater overall yield. Suitable salts, as known in the art, may be used to introduce desired metal cations to a process according to the invention. For example, halides, such as chlorides or sulphates can be used in a process according to the invention.

In some processes of the invention, the concentration of the divalent cation ranges from 0.01 mM to 500 mM. In some preferred improved enzymatic process of the invention, the divalent cation is Co²⁺ or Mn²+. In some preferred embodiments, the divalent cation is Co²⁺. In other preferred embodiments, the divalent cation is Mn²⁺. When the the divalent cation is Co²⁺, the concentration of Co²⁺ ranges from about 0.01 mM to 500 mM. For example, the concentration of Co²⁺ is about 0.1 mM. 0.2 mM, 0.5mM, 1 mM, 1.5 mM, 2 mM, 2.5 mM, 3 mM, 3.5 mM, 4 mM, 4.5 mM, 5 mM, 10 mM, 20 mM, 50 mM, 100 mM, 200 mM or 500 mM. When the divalent cation is Mn²⁺, the concentration of Mn²⁺ ranges from about 0.01 mM to 500 mM. For example, the concentration of Mn²⁺is about 0.05 mM, 0.1 mM, 0.15 mM, 0.2mM, 0.25 mM, 0.3 mM, 0.3 mM, 0.35 mM, 0.4 mM, 0.45 mM, 0.5 mM, 0.55 mM, 0.6 mM, 0.65 mM, 0.7 mM, 0.75 mM, 0.8 mM, 0.85 mM, 0.9 mM, 0.95 mM, 1 mM, 1.5 mM, 2 mM, 2.5 mM, 3 mM, 3.5 mM, 4 mM, 4.5 mM, 5 mM, 10 mM, 20 mM, 50 mM, 100 mM, 200 mM or 500 mM.

In improved enzymatic processes of the invention, the F6PP is specific for fructose, i.e., the F6PP has a higher specific activity for fructose over other sugar phosphates, such as for example glucose 6-phosphate. A non-limiting example of an F6PP is Uniprot ID B8CWV3, with the amino acid sequence set forth in SEQ ID NO: 1

(MIEAVIFDMDGVIINSEPIHYKVNQIIYEKLGIKVPRSEYNTFIGKSN
TDIWSFLKRKYNLKESVSSLIEKQISGNIKYLKSHEVNPIPGVKPLLDE
LSEKQITTGLASSSPEIYIETVLEELGLKSYFKVTVSGETVARGKPEPD
IFEKAARILGVEPPHCVVIEDSKNGVNAAKAAGMICIGYRNEESGDQDL
SAADVVVDSLEKVNYQFIKDLI).

Examples of F6PPs also include any homologues having at least 25%, at least 30%, more preferably at least 35%, more preferably at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, even more preferably at least 90%, most preferably at least 95%, at least 91%, at least 92%, at least 93%, or at least 94%, and even most preferably at least 96, 97, 98, 99 or 100% amino acid sequence identity to the aforementioned Uniprot ID.

In some preferred improved enzymatic process of the invention, a F6PP to convert F6P to fructose, contains but is not limited to containing a Rossmanoid fold domain for catalysis; additionally but not limited to containing a C1 or C2 capping domain for substrate specificity; additionally but not limited to containing a DxD signature in the 1^{st} β-strand of the Rossmanoid fold for coordinating a divalent cation where the second Asp is a general acid/base catalyst; additionally but not limited to containing a Thr or Ser at the end of the 2^{nd} β-strand of the Rossmanoid fold that helps stability of reaction intermediates; additionally but not limited to containing a Lys at the N-terminus of the a-helix C-terminal to the 3^{rd} β-strand of the Rossmanoid fold that helps stability of reaction intermediates; and additionally but not limited to containing a GDxxxD, GDxxxxD, DD, or ED signature at the end of the 4^{th} β-strand of the Rossmanoid fold for coordinating a divalent cation. These features are known in the art and are referenced in, for example, Burroughs et al., Evolutionary Genomics of the HAD Superfamily: Understanding the Structural Adaptations and Catalytic Diversity in a Superfamily of Phosphoesterases and Allied Enzymes. J. Mol. Biol. 2006; 361; 1003-1034.

Some improved enzymatic processes for preparing fructose according to the invention also include the step of enzymatically converting glucose 6-phosphate (G6P) to the F6P, catalyzed by phosphoglucoisomerase (PGI). In other embodiments, the process additionally includes the step of converting glucose 1-phosphate (G1P) to the G6P, catalyzed by phosphoglucomutase (PGM). In yet further embodiments, a fructose production process also includes the step of converting a saccharide to the G1P that is catalyzed by at least one enzyme. In some fructose production processes involving starch derivatives 4-a-glucotransferas (4GT) is added to enhance yield.

In one embodiment, an improved process for preparing fructose according to the invention, for example, includes the following steps: (i) converting a saccharide to glucose 1-phosphate (G1P) using one or more enzymes; (ii) converting G1P to G6P using phosphoglucomutase (PGM, EC 5.4.2.2); (iii) converting G6P to F6P using phosphoglucoisomerase (PGI, EC 5.3.1.9); (iv) converting F6P to fructose using F6PP in the presence of a divalent cation selected from the group consisting of Mg²⁺, Zn²⁺, Ca²⁺, Co²⁺, Mn²⁺, and combinations thereof. In another embodiment, an improved process for preparing fructose according to the invention, for example, includes the following steps: (i) converting a saccharide to glucose 1-phosphate (G1P) using one or more enzymes; (ii) converting G1P to G6P using phosphoglucomutase (PGM, EC 5.4.2.2); (iii) converting G6P to F6P using phosphoglucoisomerase (PGI, EC 5.3.1.9); (iv) converting F6P to fructose using F6PP in the presence of Mn²⁺, and a divalent cation selected from the group consisting of Zn²⁺, Ca²⁺, Co²⁺, Mn²⁺, and combinations thereof.

Typically, the ratios of enzyme units used in the disclosed process are 1:1:1:1:1 (4GT:αGP:PGM:PGI:F6PP). To optimize product yields, these ratios can be adjusted in any number of combinations. For example, a particular enzyme may be present in an amount about 2x, 3x, 4x, 5x, and so on, relative to the amount of other enzymes.

One of the important advantages of the improved processes of the invention is that the process steps can be conducted in a single bioreactor or reaction vessel. Alternatively, the steps can also be conducted in a plurality of bioreactors, or reaction vessels, that are arranged in series.

Phosphate ions produced during the dephosphorylation step can then be recycled in the process step of converting a saccharide to G1P, particularly when all process steps are conducted in a single bioreactor or reaction vessel. The ability to recycle phosphate in the disclosed processes allows for non-stoichiometric amounts of phosphate to be used, which keeps reaction phosphate concentrations low. This affects the overall pathway and the overall rate of the processes but does not limit the activity of the individual enzymes and allows for overall efficiency of the fructose production processes.

For example, reaction phosphate concentrations in each of the processes can range from about 0.1 mM to about 300 mM, from about 0 mM to about150 mM, from about 1 mM to about 50 mM, preferably from about 5 mM to about 50 mM, or more preferably from about 10 mM to about 50 mM. For instance, the reaction phosphate concentration in each of the processes can be about 0.1 mM, about 0.5 mM, about 1 mM, about 1.5 mM, about 2 mM, about 2.5 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, or about 55 mM.

Low phosphate concentration results in decreased production costs due to low total phosphate and thus lowered cost of phosphate removal. It also prevents inhibition of process enzymes by high concentrations of free phosphate and decreases the potential for phosphate pollution.

In some embodiments, the improved enzymatic processes of the invention are conducted without added ATP as a source of phosphate, i.e., ATP-free. In some embodiments the processes can also be conducted without having to add NAD(P)(H), i.e., NAD(P)(H)-free. Other advantages also include the fact that the last step of the disclosed processes for making a fructose involves an energetically favorable chemical reaction.

Methods of preparing fructose from starch and its derivatives, cellulose and its derivatives, and sucrose and its derivatives can be found, for example in PCT Pat. Appl. Pub. No. WO 2018/169957, which is incorporated by reference in its entirety.

Derivatives of starch can be prepared by enzymatic hydrolysis of starch or by acid hydrolysis of starch. Specifically, the enzymatic hydrolysis of starch can be catalyzed or enhanced by isoamylase (IA, EC. 3.2.1.68), which hydrolyzes α-1,6-glucosidic bonds; pullulanase (PA, EC. 3.2.1.41), which hydrolyzes α-1,6-glucosidic bonds; 4-α-glucanotransferase (4GT, EC. 2.4.1.25), which catalyzes the transglycosylation of short maltooligosaccharides, yielding longer maltooligosaccharides; or alpha- amylase (EC 3.2.1.1), which cleaves α-1,4-glucosidic bonds.

Furthermore, derivatives of cellulose can be prepared by enzymatic hydrolysis of cellulose catalyzed by cellulase mixtures, by acids, or by pretreatment of biomass.

Enzymes used to convert a saccharide to G1P may include αGP. For example, in a process where the saccharides include starch, the G1P is generated from starch by αGP; when the saccharides contain soluble starch, amylodextrin, or maltodextrin, the G1P is produced from soluble starch, amylodextrin, or maltodextrin and free phosphate by αGP. In some embodiments where the saccharide is maltodextrin, the maltodextrin is deashed. In other embodiments, the maltodextrin is not deashed.

When the saccharides include maltose and the enzymes contain maltose phosphorylase, the G1P is generated from maltose and free phosphate by maltose phosphorylase. If the saccharides include sucrose, and enzymes contain sucrose phosphorylase, the G1P is generated from sucrose and free phosphate by sucrose phosphorylase.

When the saccharides include cellobiose, and the enzymes contain cellobiose phosphorylase, the G1P may be produced from cellobiose by cellobiose phosphorylase. When the saccharides contain cellodextrins and the enzymes include cellodextrin phosphorylase, the G1P can be generated from cellodextrins and free phosphate by cellodextrin phosphorylase. In converting a saccharide to G1P, when the saccharides include cellulose, and enzymes contain cellulose phosphorylase, the G1P may be generated from cellulose and free phosphate by cellulose phosphorylase.

Fructose can also be produced from sucrose. Improved enzymatic process of the invention of converting sucrose to fructose include: generating G1P from sucrose and free phosphate catalyzed by sucrose phosphorylase (SP); converting G1P to G6P catalyzed by PGM; converting G6P to F6P catalyzed by PGI; converting F6P to fructose catalyzed by F6PP. The phosphate ions generated during the F6P dephosphorylation step can be recycled in the step of converting sucrose to G1P.

Improved processes of the invention include processes for converting saccharides, such as polysaccharides and oligosaccharides in starch, cellulose, sucrose and their derived products, to fructose. Artificial (non-natural) ATP-free enzymatic pathways may be provided to convert starch, cellulose, sucrose, and their derived products to fructose using cell-free enzyme cocktails.

Several enzymes can be used to hydrolyze starch to increase the G1P yield. Such enzymes include isoamylase, pullulanase, and alpha-amylase. Corn starch contains many branches that impede αGP action. Isoamylase and pullulanse can be used to de-branch starch, yielding linear amylodextrin. Isoamylase and pullulanase cleave alpha-1,6-glycosidic bonds, which allows for more complete degradation of starch by alpha-glucan phosphorylase. Alpha-amylase cleaves alpha-1,4-glycosidic bonds, therefore alpha-amylase is used to degrade starch into fragments (i.e., maltodextrin) for quicker conversion to fructose and enhanced solubility.

Maltose phosphorylase (MP) can be used to increase fructose yields by phosphorolytically cleaving the degradation product maltose into G1P and glucose. Alternatively, 4-glucan transferase (4GT) can be used to increase fructose yields by recycling the degradation products glucose, maltose, and maltotriose into longer maltooligosaccharides; which can be phosphorolytically cleaved by αGP to yield G1P.

Additionally, cellulose is the most abundant bio resource and is the primary component of plant cell walls. Non-food lignocellulosic biomass contains cellulose, hemicellulose, and lignin as well as other minor components. Pure cellulose, including Avicel (microcrystalline cellulose), regenerated amorphous cellulose, bacterial cellulose, filter paper, and so on, can be prepared via a series of treatments. The partially hydrolyzed cellulosic substrates include water-insoluble cellodextrins whose degree of polymerization is more than 7, water-soluble cellodextrins with degree of polymerization of 3-6, cellobiose, glucose, and fructose.

Cellulose and its derived products can be converted to fructose through a series of steps. The improved process of the invention also provide in vitro synthetic pathways that involves the following steps: generating G1P from cellodextrin and cellobiose and free phosphate catalyzed by cellodextrin phosphorylase (CDP) and cellobiose phosphorylase (CBP), respectively; converting G1P to G6P catalyzed by PGM; converting G6P to F6P catalyzed by PGI. In this process, the phosphate ions can be recycled by the step of converting cellodextrin and cellobiose to G1P.

Several enzymes may be used to hydrolyze solid cellulose to water-soluble cellodextrins and cellobiose. Such enzymes include endoglucanase and cellobiohydrolase, but not including beta- glucosidase (cellobiase).

Prior to cellulose hydrolysis and G1P generation, cellulose and biomass can be pretreated to increase their reactivity and decrease the degree of polymerization of cellulose chains. Cellulose and biomass pretreatment methods include dilute acid pretreatment, cellulose solvent-based lignocellulose fractionation, ammonia fiber expansion, ammonia aqueous soaking, ionic liquid treatment, and partially hydrolyzed by using concentrated acids, including hydrochloric acid, sulfuric acid, phosphoric acid and their combinations.

Polyphosphate and polyphosphate glucokinase (PPGK) can be added to the processes according to the invention, thus increasing yields of fructose by phosphorylating the degradation product glucose to G6P.

Fructose can be generated from glucose. The processes for fructose production may involve the steps of generating G6P from glucose and polyphosphate catalyzed by polyphosphate glucokinase (PPGK) and converting G6P to F6P catalyzed by PGI.

Any suitable biologically compatible buffering agent known in the art can be used in each of the processes of the invention, such as HEPES, PBS, BIS-TRIS, MOPS, DIPSO, Trizma, etc. The reaction buffer for the processes according to the invention can have a pH ranging from 5.0-8.0. More preferably, the reaction buffer pH can range from about 6.0 to about 7.3. For example, the reaction buffer pH can be 6.0, 6.2, 6.4, 6.6, 6.8, 7.0, 7.2, or 7.3.

In each of the processes of the invention the reaction temperature at which the process steps are conducted can range from 37-95° C. More preferably, the steps can be conducted at a temperature ranging from about 40° C. to about 90° C. The temperature can be, for example, about 40° C., about 45° C., about 50° C., about 55° C., about 60° C., about 65° C., about 70° C., about 75° C., about 80° C., about 85° C., or about 90° C. Preferably, the reaction temperature is about 50° C.

The reaction time of each of the improved processes for producing fructose can be adjusted as necessary, and can range for example, from about 8 hours to about 48 hours. For example, the reaction time can be about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, or about 48 hours. More preferably, the reaction time is about 24 hours. In some embodiments, fructose is produced in a continuous reaction.

Processes of the invention use low-cost starting materials and reduce production costs by decreasing costs associated with the feedstock and product separation. Starch, cellulose, sucrose and some of their derivatives are less expensive feedstocks than, for example, lactose. When fructose is produced from biomass or lactose, yields are lower than in the present invention, and fructose must be separated from other sugars via chromatography, which leads to higher production costs. Furthermore, processes of the invention are animal-free.

The step of converting F6P to fructose according to the invention is an irreversible phosphatase reaction, regardless of the feedstock. Therefore, fructose is produced with a very high yield while effectively minimizing the subsequent product separation costs.

In some embodiments, the invention involves a cell-free preparation of fructose, has relatively high reaction rates due to the elimination of the cell membrane, which often slows down the transport of substrate/product into and out of the cell. It also has a final product free of nutrient-rich fermentation media/cellular metabolites.

A particular embodiment of the invention is fructose produced by the improved processes described herein for producing fructose.

### EXAMPLES

### Materials and Methods

All chemicals were reagent grade or higher and purchased from Sigma-Aldrich (St. Louis, MO, USA) or Fisher Scientific (Pittsburgh, PA, USA), unless otherwise noted. Restriction enzymes, T4 ligase, and Phusion DNA polymerase were purchased from New England Biolabs (Ipswich, MA, USA).

Oligonucleotides were synthesized either by Integrated DNA Technologies (Coralville, IA, USA) or Eurofins MWG Operon (Huntsville, AL, USA). Escherichia coli Sig10 (Sigma-Aldrich, St. Louis, MO, USA) was used as a host cell for DNA manipulation and E. coli BL21 (DE3) (Sigma-Aldrich, St. Louis, MO, USA) was used as a host cell for recombinant protein expression. ZYM-5052 media including either 100 mg L-1 ampicillin or 50 mg L-1 kanamycin was used for E. coli cell growth and recombinant protein expression. Pullulanase (Catalog number: P1067) was purchased from Sigma-Aldrich (St. Louis, MO, USA) and produced by Novozymes (Franklinton, NC, USA). Maltose phosphorylase (Catalog number M8284) was purchased from Sigma-Aldrich. Deashed Maltodextrin DE 5 was purchased from Cargill (Minneapolis, MN, USA).

The E. coli BL21 (DE3) strain harboring a protein expression plasmid was incubated in a 1-L Erlenmeyer flask with 100 mL of ZYM-5052 media containing either 100 mg L-1 ampicillin or 50 mg L-1 kanamycin. Cells were grown at 37⁻ C with rotary shaking at 220 rpm for 16-24 hours. The cells were harvested by centrifugation at 12° C. and washed once with either 20 mM phosphate buffered saline (pH 7.5) containing 50 mM NaCI and 5 mM MgCl2 (heat precipitation and cellulose-binding module) or 20 mM phosphate buffered saline (pH 7.5) containing 300 mM NaCI and 5 mM imidazole (Nickel purification). The cell pellets were re-suspended in the same buffer and lysed by ultra-sonication (Fisherbrand™ Sonic Dismembrator Model 500; 5 s pulse on and 10 s off, total 21 min at 50% amplitude). After centrifugation, the target proteins in the supernatants were purified.

Three approaches were used to purify the various recombinant proteins. His-tagged proteins were purified by the Ni Sepharose 6 Fast Flow resin (GE Life Sciences, Marlborough, MA, USA). Fusion proteins containing a cellulose-binding module (CBM) and a self-cleavage intein were purified through high-affinity adsorption on a large surface-area regenerated amorphous cellulose. Heat precipitation at 60-95° C. for 5-30 min was used to purify hyperthermostable enzymes. The purity of the recombinant proteins was examined by sodium dodecyl sulfate polyacrylamide gel electrophoresis (SDS-PAGE).

Alpha-glucan phosphorylase (αGP) from Thermus sp. CCB_US3_UF1 (Uniprot ID G8NCCO) was used. Phosphoglucomutase (PGM) from Caldibacillus debilis (Uniprot ID A0A15OLLZ1) was used. Phosphoglucoisomerase (PGI) from Thermus thermophilus (Uniprot ID Q5SLL6) was used. The recombinant 4-alpha-glucanoltransferase from Anaerolinea thermophila was used (Uniprot E8MXP8). Fructose 6-phosphate phosphatase (F6PP) from Halothermothrix orenii (Uniprot ID B8CWV3) was used.

### EXAMPLE 1

### Testing of Divalent Cations to Determine Their Effect on Conversion of Maltodextrin to Fructose

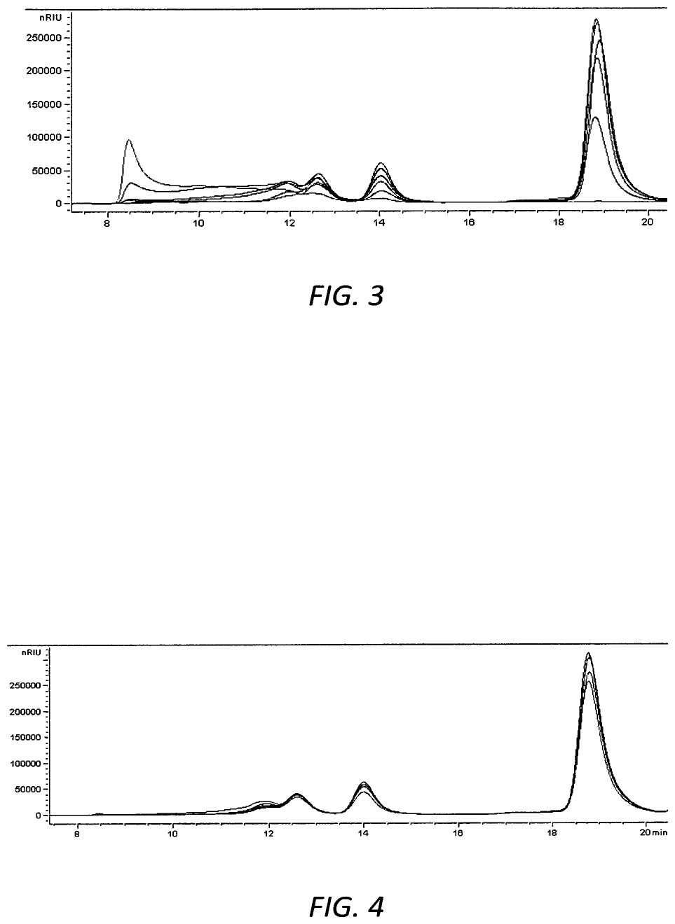

Various additional divalent cations were tested to determine their effect on the conversion of maltodextrin to fructose. A reaction of 50 mM phosphate pH 7.2, 5 mM MgC1₂, additional divalent cation (2.5 mM CaCl₂, 0.96 mM CaCl₂, 2 mM CuCl₂, 2 mM ZnCl₂, 2 mM CoCl₂, 2 mM MnCl₂), 200 g/L debranched and deashed maltodextrin, 0.8 g/L αGP, 0.1 g/L PGM, 0.1 g/L PGI, 1.0 g/L F6PP, and 0.05 g/L 4GT was prepared and incubated at 50° C. for 24 hours. The reactions were stopped via filtration of enzyme with a Vivaspin® 2 concentrator (10,000 MWCO) at 16, 20, and 24 hours. The product, fructose, was evaluated using a Supel Cogel Pb column and refractive index detector. The sample was run in ultra-pure water at 0.6 mL/min for 25 min at 80° C. The amount of fructose made in 16 hours is used to determine the relative activities of each reaction whereas the amount of fructose made in 24 hours (complete reaction) is used to determine the relative yields of each reaction. For example, at 24 hours the no additional divalent cation reaction only achieves 41% yield of fructose by weight of maltodextrin whereas the cobalt reaction achieves 88% yield of fructose by weight of maltodextrin. The results are shown in Table 1 and FIG. 3. Without wishing to be bound by any theory, it is believed that most of the benefits shown are due to enhanced stability of the F6PP.

TABLE 1

| Divalent Cation | % Activity | Relative Yield |
|---|---|---|
| Calcium | 150% | 190% |
| Zinc | 140% | 170% |
| Copper | 0% | 0% |
| Cobalt | 230% | 215% |
| Manganese | 200% | 210% |
| No additional divalent cation | 100% | 100% |
| (5 mM MgCl₂ only) | | |

### EXAMPLE 2

### Further Characterizations of Cobalt and Manganese on Conversion of Maltodextrin to Fructose

Because cobalt and manganese had the most effect, a concentration gradient study was performed the same as example 1 except for the concentrations of cobalt or manganese. The amount of fructose made in 16 hours is used to determine the relative activities of each reaction whereas the amount of fructose made in 24 hours (complete reaction) is used to determine the relative yields of each reaction. The chromatogram in FIG. 4 shows that manganese is effective at concentrations as low as 0.1 mM or lower, whereas cobalt loses effectiveness below 1 mM. 1 mM cobalt =blue; 0.5 mM cobalt =red; 0.1 mM cobalt =green; 1 mM manganese =pink; 0.5 mM manganese =gold; and 0.1 mM manganese =purple. Table 2 shows the % activity of the above reactions (16 hour time points) as well as the relative yields (24 hour time point).

TABLE 2

| [Divalent Cation] | % Activity | Relative Yield |
|---|---|---|
| 1 mM Cobalt | 210% | 245% |
| 0.5 mM Cobalt | 205% | 220% |
| 0.1 mM Cobalt | 180% | 205% |
| 1 mM Manganese | 200% | 235% |
| 0.5 mM Manganese | 200% | 235% |
| 0.1 mM Manganese | 180% | 245% |
| No additional divalent cation | 100% | 100% |
| (5 mM MgCl₂ only) | | |

### EXAMPLE 3

### Interdependence of Magnesium and Cobalt/Manganese on Conversion of Maltodextrin to Fructose

To determine the interdependence of magnesium and cobalt/manganese experiments were performed with increased amounts of magnesium or colbalt/manganese without magnesium. The reactions are the same as Example 1 except the total divalent metals are equal to the following: 25 mM MgCl₂, 1 mM CoCl₂, or 1 mM MnCl₂. The amount of fructose made in 16 hours is used to determine the relative activities of each reaction whereas the amount of fructose made in 24 hours (complete reaction) is used to determine the relative yields of each reaction. Table 3 shows the % activity of the above reactions (16 hour time points) as well as the relative yields (24 hour time point).

Taken together, the examples indicate that in general more divalent cations are beneficial to the reaction. Surprisingly, the reaction with a five-fold magnesium concentration showed % activity and relative yeild similar to the reaction with just manganese. A mixture of magnesium with manganese or cobalt is preferred for a process looking to limit conductivity or ionic strength.

TABLE 3

| [Divalent Cation] | % Activity | Relative Yield |
|---|---|---|
| 25 mM MgCl₂ Only | 140% | 185% |
| 1 mM CoCl₂ Only | 94% | 150% |
| 1 mM MnCl₂ Only | 145% | 185% |
| No additional divalent cation | 100% | 100% |
| (5 mM MgCl₂ only) | | |

SEQUENCE LISTING

SEQ ID NO. 1 Fructose 6-Phosphate Phosphatase
(Uniprot ID B8CWV3)
MIEAVIFDMDGVIINSEPIHYKVNQIIYEKLGIKVPRSEYNTFIGKSNTD
IWSFLKRKYNLKESVSSLIEKQISGNIKYLKSHEVNPIPGVKPLLDELSE
KQITTGLASSSPEIYIETVLEELGLKSYFKVTVSGETVARGKPEPDIFEK
AARILGVEPPHCVVIEDSKNGVNAAKAAGMICIGYRNEESGDQDLSAADV
VVDSLEKVNYQFIKDLI
SEQ ID NO. 2 Alpha-Glucan Phosphorylase (Uniprot
ID G8NCC0)
MPLLPEPLSGLKELAYNLWWSWNPEAAELFQEIDPSLWKRFRGNPVKLLL
EADPGRLEGLAATSYPARVGAVVEALRAYLREREEKQGPLVAYFSAEYGF
HSSLPIYSGGLGVLAGDHVKAASDLGLNLVGVGIFYHEGYFHQRLSPEGV
QVEVYETLHPEELPLYPVQDREGRPLRVGVEFPGRTLWLSAYRVQVGAVP
VYLLTANLPENTPEDRAITARLYAPGLEMRIQQELVLGLGGVRLLRALGL
APEVFHMNEGHSAFLGLERVRELVAEGHPFPVALELARAGALFTTHTPVP
AGHDAFPLELVERYLGGFWERMGTDRETFLSLGLEEKPWGKVFSMSNLAL
RTSAQANGVSRLHGEVSREMFHHLWPGFLREEVPIGHVTNGVHTWTFLHP
RLRRHYAEVFGPEWRKRPEDPETWKVEALGEEFWQIHKDLRAELVREVRT
RLYEQRRRNGESPSRLREAEKVLDPEALTIGFARRFATYKRAVLLFKDPE
RLRRLLHGHYPIQFVFAGKAHPKDEPGKAYLQELFAKIREYGLEDRMVVL
EDYDMYLARVLVHGSDVWLNTPRRPMEASGTSGMKAALNGALNLSVLDGW
WAEAYNGKNGFAIGDERVYESEEAQDMADAQALYDVLEFEVLPLFYAKGP
EGYSSGWLSMVHESLRTVGPRYSAARMVGDYLEIYRRGGAWAEAARAGQE
ALAAFHQALPALQGVTLRAQVPGDLTLNGVPMRVRAFLEGEVPEALRPFL
EVQLVVRRSSGHLEVVPMRPGPDGYEVAYRPSRPGSYAYGVRLALRHPIT
GHVAWVRWA
SEQ ID NO. 3 Phosphoglucomutase (Uniprot ID
A0A150LLZ1)
MEWKQRAERWLRFENLDPELKKQLEEMAKDEKKLEDLFYKYLEFGTGGMR
GEIGPGTNRINIYTVRKASEGLARFLLASGGEEKAKQGVVIAYDSRRKSR
EFALETAKTVGKHGIKAYVFESLRPTPELSFAVRYLHAAAGVVITASHNP
PEYNGYKVYGEDGGQLTPKAADELIRYVYEVEDELSLTVPGEQELIDRGL
LQYIGENIDLAYIEKLKTIQLNRDVILNGGKDLKIVFTPLHGTAGQLVQT
GLREFGFQNVYVVKEQEQPDPDFSTVKSPNPEEHEAFEIAIRYGKKYDAD
LIMGTDPDSDRLGIVVKNGQGDYVVLTGNQTGAILLYYLLSQKKEKGMLV
RNSAVLKTIVTSELGRAIASDFGVETIDTLTGFKFIGEKIKEFKETGSHV
FQFGYEESYGYLIGDFVRDKDAIQAALFAAEAAAYYKAQGKSLYDVLMEI
YKKYGFYKESLRSITLKGKDGAEKIRAIMDAFRQNPPEEVSGIPVAITED
YLTQKRVDKAAGQTTPIHLPKSNVLKYYLADESWFCIRPSGTEPKCKFYF
AVRGDSEAQSEARLRQLETNVMAMVEKILQK
SEQ ID NO 4 Phosphoglucoisomerase (Uniprot ID
Q5SLL6)
MLRLDTRFLPGFPEALSRHGPLLEEARRRLLAKRGEPGSMLGWMDLPEDT
ETLREVRRYREANPWVEDFVLIGIGGSALGPKALEAAFNESGVRFHYLDH
VEPEPILRLLRTLDPRKTLVNAVSKSGSTAETLAGLAVFLKWLKAHLGED
WRRHLVVTTDPKEGPLRAFAEREGLKAFAIPKEVGGRFSALSPVGLLPLA
FAGADLDALLMGARKANETALAPLEESLPLKTALLLHLHRHLPVHVFMVY
SERLSHLPSWFVQLHDESLGKVDRQGQRVGTTAVPALGPKDQHAQVQLFR
EGPLDKLLALVIPEAPLEDVEIPEVEGLEAASYLFGKTLFQLLKAEAEAT
YEALAEAGQRVYALFLPEVSPYAVGWLMQHLMWQTAFLGELWEVNAFDQP
GVELGKVLTRKRLAG
SEQ ID NO 5 4-Glucan Transferase (Uniprot ID
E8MXP8)
MSLFKRASGILLHPTSLPGPDGIGDLGPEAYRWVNFLAESGCSLWQILPL
GPTGFGDSPYQCFSAFAGNPYLVSPALLLDEGLLTSEDLADRPEFPASRV
DYGPVIQWKLTLLDRAYVRFKRSTSQKRKAAFEAFKEEQRAWLLDFSLFM
AIKEAHGGASWDYWPEPLRKRDPEALNAFHRAHEVDVERHSFRQFLFFRQ
WQALRQYAHEKGVQIIGDVPIFVAYDSADVWSHPDLFYLDETGKPTVVAG
VPPDYFSATGQLWGNPLYRWDYHRETGFAWWLERLKATFAMVDIVRLDHF
RGFAGYWEVPYGMPTAEKGRWVPGPGIALFEAIRNALGGLPIIAEDLGEI
TPDVIELREQLGLPGMKIFQFAFASDADDPFLPHNYVQNCVAYTGTHDND
TAIGWYNSAPEKERDFVRRYLARSGEDIAWDMIRAVWSSVAMFAIAPLQD
FLKLGPEARMNYPGRPAGNWGWRYEAFMLDDGLKNRIKEINYLYGRLPEH
MKPPKVVKKWT

1. A process for preparing fructose from a saccharide comprising:
a step of converting fructose 6-phosphate (F6P) to a fructose catalyzed by fructose 6-phosphate phosphatase (F6PP) in the presence of a divalent cation selected from the group consisting of Mg²⁺, Zn²⁺, Ca²⁺, Co²⁺, Mn²⁺, and combinations thereof.

2. The process of claim 1, wherein the step of converting F6P to fructose catalyzed by F6PP is in the presence of Mg²⁺ and a divalent cation selected from the group consisting of Zn²⁺, Ca²⁺, Co²⁺, Mn²⁺, and combinations thereof.

3. The process of claim 1, wherein the concentration of the divalent cation ranges from 0.01 mM to 500 mM.

4. The process of claim 1, wherein the divalent cation is Co²⁺or Mn^{2+.}

5. The process of claim 1, wherein the divalent cation is Co²⁺.

6. The process of claims 1, wherein the divalent cation is Mn²⁺.

7. The process of claim 1, further comprising a step of converting glucose 6-phosphate (G6P) to the F6P, wherein the step is catalyzed by phosphoglucoisomerase (PGI).

8. The process of claim 7, further comprising the step of converting glucose 1-phosphate (G1P) to the G6P, wherein the step is catalyzed by phosphoglucomutase (PGM).

9. The process of claim 1, wherein the saccharide is selected from sucrose, starch, or a starch derivative selected from the group consisting of amylose, amylopectin, soluble starch, amylodextrin, maltotriose, maltodextrin, maltose, and glucose.

10. The process of claim 9, further comprising the step of converting the saccharide to the G1P, wherein the step is catalyzed by at least one enzyme.

11. The process of claim 10, wherein the at least one enzyme in the step of converting a saccharide to the G1P is selected from the group consisting of alpha-glucan phosphorylase (αGP), maltose phosphorylase, and sucrose phosphorylase, and mixtures thereof.

12. The process of claim 11, wherein the saccharide is starch, further comprising the step of converting starch to a starch derivative wherein the starch derivative is prepared by enzymatic hydrolysis of starch or by acid hydrolysis of starch.

13. The process of claim 11, wherein 4-glucan transferase (4GT) is added to the process.

14. The process of claim 12, wherein the starch derivative is prepared by enzymatic hydrolysis of starch catalyzed by isoamylase, pullulanase, alpha-amylase, or a combination thereof.

15. The process of claim 1, wherein the process steps are conducted at a temperature ranging from about 37° C. to about 95° C., at a pH ranging from about 5.0 to about 8.0, and/or for about 8 hours to about 48 hours.

16. The process of claim 1, wherein the process steps are conducted in a single bioreactor.

17. The process of claim 1, wherein the process steps are conducted ATP-free, NAD(P)(H)-free, at a phosphate concentration from about 0.1 mM to about 150 mM, the phosphate is recycled, and/or at least one step of the process involves an energetically favorable chemical reaction.
